(19) United States
(12) Patent Application Publication
(10) Pub. No.: US 2006/0199176 A1
(43) Pub. Date: Sep. 7, 2006

(54) Coronavirus S peptides

(76) Inventors: Yeau-Ching Wang, Taipei (TW); Szu-Chia Lai, Taipei (TW); Chia-Tsui Yeh, Taipei (TW); Pele Choi Sing Chong, Richmond Hill (CA); Shih-Jen Liu, Taipei County (TW)

Correspondence Address: FISH & RICHARDSON PC, P.O. BOX 1022, MINNEAPOLIS, MN, 55440-1022, US

(21) Appl. No.: 11/181,693

(22) Filed: Jul. 14, 2005

Related U.S. Application Data
(60) Provisional application No. 60/588,087, filed on Jul. 15, 2004.

(51) Int. Cl. C12Q 1/70; C12N 5/06; C12N 15/86; C07K 14/165; C07K 16/12

(52) U.S. Cl. 435/5; 435/69.3; 435/456; 435/325; 530/350; 530/324; 530/325; 530/327; 530/328; 530/329; 530/330; 530/388.3

(57) ABSTRACT

Isolated polypeptides containing SEQ ID NO: 3 and functional equivalents thereof. Also disclosed are isolated nucleic acids encoding the polypeptides, related expression vectors, related host cells, related antibodies, and related compositions. Methods of producing the polypeptide, diagnosing infection with a coronavirus, and identifying a test compound for treating infection with a coronavirus are also disclosed.

### RELATED APPLICATION

This application claims priority to U.S. Provisional Application Ser. No. 60/588,087, filed on Jul. 15, 2004, the content of which is incorporated by reference in its entirety.

### BACKGROUND

Virus is the cause of various disorders. For example, members of the coronavirus (CoV) family cause hepatitis in mice, gastroenteritis in pigs, and respiratory infections in birds and humans. Among the more than 30 strains isolated so far, three or four infect humans. The severe acute respiratory syndrome (SARS), a newly found infectious disease, is associated with a novel coronavirus. This life-threatening respiratory coronavirus touched off worldwide outbreaks in 2003. There is a need for a method of diagnosing infection with SARS virus and a drug for treating the infection.

### SUMMARY

This invention is based, at least in part, on the discovery of a neutralization epitope in the SARS CoV Spike (S) protein. Listed below are the amino acid (aa) sequence of the SARS CoV Urbani strain S protein (SEQ ID NO: 1) and the nucleotide (nt) sequence encoding it (SEQ ID NO: 2). SEQ ID NO: 2 corresponds to nt 21,492-25,259 of GenBank Accession No. AY278741.

SEQ ID NO: 1
MFIFLLFLTLTSGSDLDRCTTFDDVQAPNYTQHTSSMRGVYYPDEIFRSDTLYLTQDLFLPFYSNVTGFHT
INHTFGNPVIPFKDGIYFAATEKSNVVRGWVFGSTMNNKSQSVIIINNSTNVVIRACNFELCDNPFFAVSK
PMGTQTHTMIFDNAFNCTEEYISDAFSLDVSEKSGNFKHLREFVFKNKDGFLYVYKGYQPIDVVRDLPSGF
NTLKPIFKLPLGINITNFRAILTAFSPAQDIWGTSAAAYFVGYLKPTTFMLKYDENGTITDAVDCSQNPLA
ELKCSVKSFEIDKGIYQTSNFRVVPSGDVVRFPNITNLCPFGEVFNATKFPSVYAWERKKISNCVADYSVL
YNSTFFSTFKCYGVSATKLNDLCFSNVYADSFVVKGDDVRQIAPGQTGVIADYNYKLPDDFMGCVLAWNTR
NIDATSTGNYNYKYRYLRHGKLRPFERDISNVPFSPDGKPCTPPALNCYWPLNDYGFYTTTGIGYQPYRVV
VLSFELLNAPATVCGPKLSTDLIKNQCVNFNFNGLTGTGVLTPSSKRFQPFQQFGRDVSDFTDSVRDPKTS
EILDISPCAFGGVSVITPGTNASSEVAVLYQDVNCTDVSTAIHADQLTPAWRIYSTGNNVEQTQAGCLICA
EHVDTSYECDIPIGAGICASYHTVSLLRSTSQKSIVAYTMSLGADSSIAYSNNTIAIPTNFSISITTEVMP
VSMAKTSVDCNMYICGDSTECANLLLQYGSFCTQLNRALSGIAAEQDRNTREVFAQVKQMYKTPTLKYFGG
FNFSQILPDPLKPTKRSFIEDLLFNKVTLADAGFMKQYGECLGDINARDLICAQKFNGLTVLPPLLTDDMI
AAYTAALVSGTATAGWTFGAGAALQIPFAMQMAYRFNGIGVTQNVLYENQKQIANQFNKAISQIQESLTTT
STALGKLQDVVNQNAQALNTLVKQLSSNFGAISSVLNDILSRLDKVEAEVQIDRLITGRLQSLQTYVTQQL
IRAAEIRASANLAATKMSECVLGQSKRVDFCGKGYHLMSFPQAAPHGVVFLHVTYVPSQERNFTTAPAICH
EGKAYFPREGVFVFNGTSWFITQRNFFSPQIITTDNTFVSGNCDVVIGIINNTVYDPLQPELDSFKEELDK
YFKNHTSPDVDLGDISGINASVVNIQKEIDRLNEVAKNLNESLIDLQELGKYEQYIKWPWYVWLGFIAGLI
AIVMVTILLCCMTSCCSCLKGACSCGSCCKFDEDDSEPVLKGVKLHYT
SEQ ID NO: 2
21492 atgtttatt ttcttattat ttcttactct cactagtggt agtgaccttg
21541 accggtgcac cacttttgat gatgttcaag ctcctaatta cactcaacat acttcatcta
21601 tgaggggggt ttactatcct gatgaaattt ttagatcaga cactctttat ttaactcagg
21661 atttatttct tccattttat tctaatgtta cagggtttca tactattaat catacgtttg
21721 gcaaccctgt catacctttt aaggatggta tttattttgc tgccacagag aaatcaaatg
21781 ttgtccgtgg ttgggttttt ggttctacca tgaacaacaa gtcacagtcg gtgattatta
21841 ttaacaattc tactaatgtt gttatacgag catgtaactt tgaattgtgt gacaaccctt
21901 tctttgctgt ttctaaaccc atgggtacac agacacatac tatgatattc gataatgcat
21961 ttaattgcac tttcgagtac atatctgatg ccttttcgct tgatgtttca gaaaagtcag
22021 gtaattttaa acacttacga gagtttgtgt ttaaaaataa agatgggttt ctctatgttt
22081 ataagggcta tcaacctata gatgtagttc gtgatctacc ttctggtttt aacactttga
22141 aacctatttt taagttgcct cttggtatta acattacaaa ttttagagcc attcttacag
22201 ccttttcacc tgctcaagac atttggggca cgtcagctgc agcctatttt gttggctatt
22261 taaagccaac tacatttatg ctcaagtatg atgaaaatgg tacaatcaca gatgctgttg
22321 attgttctca aaatccactt gctgaactca aatgctctgt taagagcttt gagattgaca
22381 aaggaattta ccagacctct aatttcaggg ttgttccctc aggagatgtt gtgagattcc
22441 ctaatattac aaacttgtgt ccttttggag aggtttttaa tgctactaaa ttcccttctg
22501 tctatgcatg ggagagaaaa aaaatttcta attgtgttgc tgattactct gtgctctaca
22561 actcaacatt tttttcaacc tttaagtgct atggcgtttc tgccactaag ttgaatgatc
22621 tttgcttctc caatgtctat gcagattctt ttgtagtcaa gggagatgat gtaagacaaa
22681 tagcgccagg acaaactggt gttattgctg attataatta taaattgcca gatgatttca
22741 tgggttgtgt ccttgcttgg aatactagga acattgatgc tacttcaact ggtaattata
22801 attataaata taggtatctt agacatggca agcttaggcc ctttgagaga gacatatcta
22861 atgtgccttt ctcccctgat ggcaaacctt gcaccccacc tgctcttaat tgttattggc
22921 cattaaatga ttatggtttt tacaccacta ctggcattgg ctaccaacct tacagagttg
22981 tagtactttc ttttgaactt ttaaatgcac cggccacggt ttgtggacca aaattatcca
23041 ctgaccttat taagaaccag tgtgtcaatt ttaattttaa tggactcact ggtactggtg
23101 tgttaactcc ttcttcaaag agatttcaac catttcaaca atttggccgt gatgtttctg
23161 atttcactga ttccgttcga gatcctaaaa catctgaaat attagacatt tcaccttgct
23221 cttttggggg tgtaagtgta attacacctg gaacaaatgc ttcatctgaa gttgctgttc
23281 tatatcaaga tgttaactgc actgatgttt ctacagcaat tcatgcagat caactcacac
23341 cagcttggcg catatattct actggaaaca atgtattcca gactcaagca ggctgtctta
23401 taggagctga gcatgtcgac acttcttatg agtgcgacat tcctattgga gctggcattt
23461 gtgctagtta ccatacagtt tctttattac gtagtactag ccaaaaatct attgtggctt
23521 atactatgtc tttaggtgct gatagttcaa ttgcttactc taataacacc attgctatac
23581 ctactaactt ttcaattagc attactacag aagtaatgcc tgtttctatg gctaaaacct
23641 ccgtagattg taatatgtac atctgcggag attctactga atgtgctaat ttgcttctcc
23701 aatatggtag cttttgcaca caactaaatc gtgcactctc aggtattgct gctgaacagg
23761 atcgcaacac acgtgaagtg ttcgctcaag tcaaacaaat gtacaaaacc ccaactttga
23821 aatattttgg tggttttaat ttttcacaaa tattacctga ccctctaaag ccaactaaga
23881 ggtcttttat tgaggacttg ctctttaata aggtgacact cgctgatgct ggcttcatga
23941 agcaatatgg cgaatgccta ggtgatatta atgctagaga tctcatttgt gcgcagaagt
24001 tcaatggact tacagtgttg ccacctctgc tcactgatga tatgattgct gcctacactg
24061 ctgctctagt tagtggtact gccactgctg gatggacatt tggtgctggc gctgctcttc
24121 aaataccttt tgctatgcaa atggcatata ggttcaatgg cattggagtt acccaaaatg
24181 ttctctatga gaaccaaaaa caaatcgcca accaatttaa caaggcgatt agtcaaattc
24241 aagaatcact tacaacaaca tcaactgcat tgggcaagct gcaagacgtt gttaaccaga
24301 atgctcaagc attaaacaca cttgttaaac aacttagctc taattttggt gcaatttcaa
24361 gtgtgctaaa tgatatcctt tcgcgacttg ataaagtcga ggcggaggta caaattgaca
24421 ggttaattac aggcagactt caaagccttc aaacctatgt aacacaacaa ctaatcaggg
24481 ctgctgaaat cagggcttct gctaatcttg ctgctactaa aatgtctgag tgtgttcttg
24541 gacaatcaaa aagagttgac ttttgtggaa agggctacca ccttatgtcc ttcccacaag
24601 cagccccgca tggtgttgtc ttcctacatg tcacgtatgt gccatcccag gagaggaact
24661 tcaccacagc gccagcaatt tgtcatgaag gcaaagcata cttccctcgt gaaggtgttt
24721 ttgtgtttaa tggcacttct tggtttatta cacagaggaa cttcttttct ccacaaataa
24781 ttactacaga caatacattt gtctcaggaa attgtgatgt cgttattggc atcattaaca
24841 acacagttta tgatcctctg caacctgagc tcgactcatt caaagaagag ctggacaagt
24901 acttcaaaaa tcatacatca ccagatgttg atcttggcga catttcaggc attaacgctt
24961 ctgtcgtcaa cattcaaaaa gaaattgacc gcctcaatga ggtcgctaaa aatttaaatg
25021 aatcactcat tgaccttcaa gaattgggaa aatatgagca atatattaaa tggccttggt
25081 atgtttggct cggcttcatt gctggactaa ttgccatcgt catggttaca atcttgcttt
25141 gttgcatgac tagttgttgc agttgcctca agggtgcatg ctcttgtggt tcttgctgca
25201 agtttgatga ggatgactct gagccagttc tcaagggtgt caaattacat tacacataa

One aspect of the invention features an isolated polypeptide containing SPDVDLGDISGINAS (SEQ ID NO: 3), which corresponds to aa 1143-1157 of SEQ ID NO: 1. The polypeptide is 15-100 (i.e., any integer number between 15 and 100, inclusive) amino acid residues in length. It can be 15-50 or 15-32 amino acid residues in length. One example of the polypeptide is DSFKEELDRYFKNHTSPDVDLGDISGIN-ASVV (SEQ ID NO: 4).

An “isolated polypeptide” refers to a polypeptide substantially free from naturally associated molecules, i.e., it is at least 75% (i.e., any number between 75% and 100%, inclusive) pure by dry weight. Purity can be measured by any appropriate standard method, for example, by column chromatography, polyacrylamide gel electrophoresis, or HPLC analysis. An isolated polypeptide of the invention can be purified from a natural source, produced by recombinant DNA techniques, or by chemical methods.

The invention also features an isolated nucleic acid that contains a sequence encoding one of the above-mentioned polypeptides or a complement thereof. Examples of the nucleic acid includes nt 24,918-24,962 and nt 24,873-24,968 of SEQ ID NO: 1 (SEQ ID NOs: 5 and 6), which encode SEQ ID NOs: 3 and 4, respectively. A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” is a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the polypeptide of this invention. For this purpose, one can operatively linked the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector of this invention includes a nucleic acid in a form suitable for expression of the nucleic acid in a host cell. Preferably the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vector can be introduced into host cells to produce the polypeptide of this invention.

Also within the scope of this invention is a host cell that contains the above-described nucleic acid. Examples include E. coli cells, insect cells (e.g., using baculovirus expression vectors), yeast cells, or mammalian cells. See e.g., Goeddel, (1990) Gene Expression Technology: Methods in Enzymology 185, Academic Press, San Diego, Calif. To produce a polypeptide of this invention, one can culture a host cell in a medium under conditions permitting expression of the polypeptide encoded by a nucleic acid of this invention, and purify the polypeptide from the cultured cell or the medium of the cell. Alternatively, the nucleic acid of this invention can be transcribed and translated in vitro, for example, using T7 promoter regulatory sequences and T7 polymerase.

A polypeptide and a nucleic acid of this invention can be used to induce an immune response (i.e., the production of specific antibodies) in a subject against a coronavirus by administering to the subject an effective amount of the polypeptide or nucleic acid encoding the polypeptide. They also can be used to generate specific antibodies that bind specifically to the above-described polypeptides or its fragment. More specifically, one can generate the antibodies by administering to a non-human animal the polypeptide or nucleic acid. Thus, within the scope of this invention is a composition containing the afore-mentioned polypeptide or nucleic acid; and a pharmaceutically acceptable carrier. The composition can be used to generate the antibodies. One can purify the antibodies from the subject or the non-human animal and generate monoclonal antibodies by standard techniques.

The invention features a purified antibody that binds specifically to the above-described polypeptide. The antibody can be a polyclonal or a monoclonal antibody (MAb). Examples of the monoclonal antibody include those described in the examples below, such as MAb 1, MAb 2, MAb 3, MAb 4, MAb 5, MAb 6, MAb 7, MAb 8, MAb 9, MAb 10, MAb 11, MAb 12, MAb13, MAb 1-1, MAb 3-2, MAb 5-1, and MAb 8-1.

One can use the antibodies to diagnose an infection with a coronavirus, e.g., SARS-CoV, in a subject by determining the presence of a polypeptide containing the sequence of SEQ ID NO: 3 or an immunogenic fragment thereof in a test sample (e.g., a blood sample) from the subject. Presence of the polypeptide in the test sample indicates the subject is infected with the coronavirus. One can also diagnose an infection with a coronavirus in a subject by determining presence of a specific antibody against a polypeptide having the sequence of SEQ ID NO: 3 or an immunogenic fragment thereof in the test sample. Presence of the antibody in the test sample also indicates the subject is infected with the coronavirus.

Within the scope of this invention is a method of treating an infection with a coronavirus. The method includes administering to a subject in need thereof an effective amount of one or more of the above-described polypeptides or antibodies. The term “treating” is defined as administration of a composition to a subject with the purpose to cure, alleviate, relieve, remedy, prevent, or ameliorate a disorder, the symptom of the disorder, the disease state secondary to the disorder, or the predisposition toward the disorder. An “effective amount” is an amount of the composition that is capable of producing a medically desirable result, e.g., as described above, in a treated subject.

Also within the scope of this invention is a kit for detecting presence of a coronavirus in a sample. The kit contains the above-described polypeptide or antibody. It can also contain a container for receiving the polypeptide or antibody and other reagents for immune assays.

The invention features a screening method of identifying a candidate compound (e.g., an antibody) for treating an infection with a coronavirus. The method includes incubating a test compound and a first polypeptide containing SEQ ID NO: 3; and determining binding between the test compound and the first polypeptide. Presence of the binding indicates that the compound is a candidate compound. The screening method can further include (i) incubating the test compound and a second polypeptide that is identical to the first polypeptide except that Asp₅ of SEQ ID NO: 3 is replaced with a non-Asp residue or Ser₁₀ of SEQ ID NO: 3 is replaced with a non-Ser residue; and (2) determining binding between the test compound and the second polypeptide. The compound is determined to be a candidate compound if the binding between the test compound and the first polypeptide is stronger than the binding between the test compound and the second polypeptide.

The details of one or more embodiments of the invention are set forth in the description below. Other features, objects, and advantages of the invention will be apparent from the description and from the claims.

### DETAILED DESCRIPTION

This invention relates to polypeptides containing a neutralization epitope (SEQ ID NO: 3) of the S protein of a coronavirus, such as a SARS CoV Since this epitope mediates target cell binding and entry of the coronavirus, it can be targeted for diagnosing or treating an infection with the coronavirus. For example, a polypeptide containing the neutralization epitope, via competition, inhibits the entry of SARS-CoV into its host cells and thereby neutralize the viral infection. A compound that binds to the neutralization epitope, by masking the epitope on SARS-CoV, also inhibits the entry of SARS-CoV into its host cells.

Within the scope of this invention are polypeptides containing SEQ ID NO: 3, its functional equivalent, or the equivalent sequence from the S protein of SARS CoV TW1, Tor-2, SIN2500, SIN2774, SIN2748, SIN2677, SIN2679, CUHK-W1, HKU39849, GZ01, BJ01, BJ02, BJ03 BJ04, or other strains. A functional equivalent of SEQ ID NO: 3 refers to a polypeptide derived from SEQ ID NO: 3, e.g., a fusion polypeptide or a polypeptide having one or more point mutations, insertions, deletions, truncations, or a combination thereof. In particular, such functional equivalents include polypeptides, whose sequences differ from SEQ ID NO: 3 by one or more conservative amino acid substitutions or by one or more non-conservative amino acid substitutions, deletions, or insertions. All of the just-mentioned functional equivalents or equivalent sequences have substantially the activity to mediate coronavirus's binding to and entry into host cells, e.g., VERO E6. This activity can be determined by a neutralization assay or entry-inhibition assay similar to those described in the examples below. In these assays, a functional equivalent, via competition, inhibits the entry of SARS-CoV into the host cells and thereby neutralize the viral infection.

A polypeptide of the invention can be obtained as a synthetic polypeptide or a recombinant polypeptide. To prepare a recombinant polypeptide, a nucleic acid encoding it can be linked to another nucleic acid encoding a fusion partner, e.g., Glutathione-S-Transferase (GST), 6×-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention.

A polypeptide of the invention can be used to generate antibodies in animals (for production of antibodies) or humans (for treatment of diseases). Methods of making monoclonal and polyclonal antibodies and fragments thereof in animals are known in the art. See, for example, Harlow and Lane, (1988) Antibodies: A Laboratory Manual, Cold Spring Harbor Laboratory, New York. The term “antibody” includes intact molecules as well as fragments thereof, such as Fab, F(ab′)₂, Fv, scFv (single chain antibody), and dAb (domain antibody; Ward, et. al. (1989) Nature, 341, 544). These antibodies can be used for detecting the S polypeptide, e.g., in determining whether a test sample from a subject contains coronavirus or in identifying a compound that binds to the polypeptide. As these antibodies interfere with the cell binding and entry of the coronavirus, they are also useful for treating a coronavirus infection.

In general, to produce antibodies against a polypeptide, the polypeptide can be coupled to a carrier protein, such as KLH, mixed with an adjuvant, and injected into a host animal. Antibodies produced in the animal can then be purified by peptide affinity chromatography. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, CpG, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

Polyclonal antibodies, heterogeneous populations of antibody molecules, are present in the sera of the immunized subjects. Monoclonal antibodies, homogeneous populations of antibodies to a polypeptide of this invention, can be prepared using standard hybridoma technology (see, for example, Kohler et al. (1975) Nature 256, 495; Kohler et al. (1976) Eur. J. Immunol. 6, 511; Kohler et al. (1976) Eur J Immunol 6, 292; and Hammerling et al. (1981) Monoclonal Antibodies and T Cell Hybridomas, Elsevier, N.Y.). In particular, monoclonal antibodies can be obtained by any technique that provides for the production of antibody molecules by continuous cell lines in culture such as described in Kohler et al. (1975) Nature 256, 495 and U.S. Pat. No. 4,376,110; the human B-cell hybridoma technique (Kosbor et al. (1983) Immunol Today 4, 72; Cole et al. (1983) Proc. Natl. Acad. Sci. USA 80, 2026, and the EBV-hybridoma technique (Cole et al. (1983) Monoclonal Antibodies and Cancer Therapy, Alan R. Liss, Inc., pp. 77-96). Such antibodies can be of any immunoglobulin class including IgG, IgM, IgE, IgA, IgD, and any subclass thereof. The hybridoma producing the monoclonal antibodies of the invention may be cultivated in vitro or in vivo. The ability to produce high titers of monoclonal antibodies in vivo makes it a particularly useful method of production.

In addition, techniques developed for the production of “chimeric antibodies” can be used. See, e.g., Morrison et al. (1984) Proc. Natl. Acad. Sci. USA 81, 6851; Neuberger et al. (1984) Nature 312, 604; and Takeda et al. (1984) Nature 314:452. A chimeric antibody is a molecule in which different portions are derived from different animal species, such as those having a variable region derived from a murine monoclonal antibody and a human immunoglobulin constant region. Alternatively, techniques described for the production of single chain antibodies (U.S. Pat. Nos. 4,946,778 and 4,704,692) can be adapted to produce a phage library of single chain Fv antibodies. Single chain antibodies are formed by linking the heavy and light chain fragments of the Fv region via an amino acid bridge. Moreover, antibody fragments can be generated by known techniques. For example, such fragments include, but are not limited to, F(ab′)₂ fragments that can be produced by pepsin digestion of an antibody molecule, and Fab fragments that can be generated by reducing the disulfide bridges of F(ab′)₂ fragments. Antibodies can also be humanized by methods known in the art. For example, monoclonal antibodies with a desired binding specificity can be commercially humanized (Scotgene, Scotland; and Oxford Molecular, Palo Alto, Calif.). Fully human antibodies, such as those expressed in transgenic animals are also features of the invention (see, e.g., Green et al. (1994) Nature Genetics 7, 13; and U.S. Pat. Nos. 5,545,806 and 5,569,825).

A polypeptide of the invention can also be used to prepare an immunogenic composition (e.g., a vaccine) for generating antibodies against coronavirus (e.g., SRAS CoV) in a subject susceptible to the coronavirus. Such compositions can be prepared, e.g., according to the method described in the examples below, or by any other equivalent methods known in the art. The composition contains an effective amount of a polypeptide of the invention, and a pharmaceutically acceptable carrier such as phosphate buffered saline or a bicarbonate solution. The carrier is selected on the basis of the mode and route of administration, and standard pharmaceutical practice. Suitable pharmaceutical carriers and diluents, as well as pharmaceutical necessities for their use, are described in Remington's Pharmaceutical Sciences. An adjuvant, e.g., a cholera toxin, Escherichia coli heat-labile enterotoxin (LT), liposome, immune-stimulating complex (ISCOM), or immunostimulatory sequences oligodeoxynucleotides (ISS-ODN), can also be included in a composition of the invention, if necessary. The S protein, fragments or analogs thereof or peptides may be components of a multivalent composition of vaccine against respiratory diseases. This multivalent composition contains at least one immunogenic fragment of S protein described above, along with at least one protective antigen isolated from influenza virus, para-influenza virus 3, Strentococcus pneumoniae, Branhamella (Moroxella) gatarhalis, Staphylococcus aureus, or respiratory syncytial virus, in the presence or absence of adjuvant.

Methods for preparing vaccines are generally well known in the art, as exemplified by U.S. Pat. Nos. 4,601,903; 4,599,231; 4,599,230; and 4,596,792. Vaccines may be prepared as injectables, as liquid solutions or emulsions. The S protein, fragments or analogs thereof or peptides corresponding to portions of S protein may be mixed with physiologically acceptable and excipients compatible. Excipients may include, water, saline, dextrose, glycerol, ethanol, and combinations thereof. The vaccine may further contain minor amounts of auxiliary substances such as wetting or emulsifying agents, pH buffering agents, or adjuvants to enhance the effectiveness of the vaccines. Methods of achieving adjuvant effect for the vaccine includes use of agents, such as aluminum hydroxide or phosphate (alum), commonly used as 0.05 to 0.1 percent solutions in phosphate buffered saline. Vaccines may be administered parenterally, by injection subcutaneously or intramuscularly. Alternatively, other modes of administration including suppositories and oral formulations may be desirable. For suppositories, binders and carriers may include, for example, polyalkalene glycols or triglycerides. Oral formulations may include normally employed incipients such as, for example, pharmaceutical grades of saccharine, cellulose, magnesium carbonate and the like. These compositions take the form of solutions, suspensions, tablets, pills, capsules, sustained release formulations or powders and contain 10-95% of the S protein, fragment analogs, or peptides.

The vaccines are administered in a manner compatible with the dosage formulation, and in an amount that is therapeutically effective, protective and immunogenic. The quantity to be administered depends on the subject to be treated, including, for example, the capacity of the individual's immune system to synthesize antibodies, and if needed, to produce a cell-mediated immune response. Precise amounts of active ingredient required to be administered depend on the judgment of the practitioner. However, suitable dosage ranges are readily determinable by one skilled in the art and may be of the order of micrograms of the polypeptide of this invention. Suitable regimes for initial administration and booster doses are also variable, but may include an initial administration followed by subsequent administrations. The dosage of the vaccine may also depend on the route of administration and varies according to the size of the host.

Use of polypeptide in vivo may first require chemical modification of the peptides since they may not have a sufficiently long half-life. A chemically modified peptide or a peptide analog includes any functional chemical equivalent of the peptide characterized by its increased stability and/or efficacy in vivo or in vitro in respect of the practice of the invention. The term peptide analog also refers to any amino acid derivative of a peptide as described herein. A peptide analog can be produced by procedures that include, but are not limited to, modifications to side chains, incorporation of unnatural amino acids and/or their derivatives during peptide synthesis and the use of cross-linkers and other methods that impose conformational constraint on the peptides or their analogs. Examples of side chain modifications include modification of amino groups, such as by reductive alkylation by reaction with an aldehyde followed by reduction with NaBH₄; amidation with methylacetimidate; acetylation with acetic anhydride; carbamylation of amino groups with cyanate; trinitrobenzylation of amino groups with 2, 4, 6, trinitrobenzene sulfonic acid (TNBS); alkylation of amino groups with succinic anhydride and tetrahydrophthalic anhydride; and pyridoxylation of lysine with pyridoxa-5′-phosphate followed by reduction with NABH₄. The guanidino group of arginine residues may be modified by the formation of heterocyclic condensation products with reagents such as 2,3-butanedione, phenylglyoxal and glyoxal. The carboxyl group may be modified by carbodiimide activation via o-acylisourea formation followed by subsequent derivatization, for example, to a corresponding amide. Sulfhydryl groups may be modified by methods, such as carboxymethylation with iodoacetic acid or iodoacetamide; performic acid oxidation to cysteic acid; formation of mixed disulphides with other thiol compounds; reaction with maleimide; maleic anhydride or other substituted maleimide; formation of mercurial derivatives using 4-chloromercuribenzoate, 4-chloromercuriphenylsulfonic acid, phenylmercury chloride, 2-chloromercuric-4-nitrophenol and other mercurials; carbamylation with cyanate at alkaline pH. Tryptophan residues may be modified by, for example, oxidation with N-bromosuccinimide or alkylation of the indole ring with 2-hydroxy-5-nitrobenzyl bromide or sulphonyl halides. Tryosine residues may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate. Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

A nucleic acid molecule of this invention may also be used directly for immunization by administration of the nucleic acid directly to a subject via a live vector, such as Salmonella, BCG, adenovirus, poxvirus or vaccinia. Immunization methods based on nucleic acids are well known in the art.

A subject susceptible to coronavirus infection can be identified and administered a polypeptide-containing composition of the invention. The dose of the composition depends, for example, on the particular polypeptide, whether an adjuvant is co-administered with the polypeptide, the type of adjuvant co-administered, the mode and frequency of administration, as can be determined by one skilled in the art. Administration is repeated as necessary, as can be determined by one skilled in the art. For example, a priming dose can be followed by three booster doses at weekly intervals. A booster shot can be given at 4 to 8 weeks after the first immunization, and a second booster can be given at 8 to 12 weeks, using the same formulation. Sera or T-cells can be taken from the subject for testing the immune response elicited by the composition against the coronavirus S protein or infection. Methods of assaying antibodies or cytotoxic T cells against a protein or infection are well known in the art. Additional boosters can be given as needed. By varying the amount of polypeptide, the dose of the composition, and frequency of administration, the immunization protocol can be optimized for eliciting a maximal immune response. Before a large scale administering, efficacy testing is desirable. In an efficacy testing, a non-human subject can be administered via an oral or parenteral route with a composition of the invention. After the initial administration or after optional booster administration, both the test subject and the control subject (receiving mock administration) are challenged with an LD₉₅ dose of a coronavirus. End points other than lethality can also be used. Efficacy is determined if subjects receiving the composition dies at a rate lower than control subjects. The difference in death rates should be statistically significant.

The above-described polypeptides can be used as a carrier and linked to other antigens of interest to generate antibodies against the antigens. The polypeptides fragment can be generally utilized to prepare chimeric molecules and conjugate compositions against pathogenic bacteria, including encapsulated bacteria. For example, the glycoconjugates of the present inventions may be applied to immunize a subject to generate antibodies against the bacteria and confer protection against infection with any bacteria having polysaccharide antigens, e.g., Haemophilus influenzae, Streptococcus pneumoniae, Escherichia coli, Neisseria meningitidis, Salmonella typhi, Streptococcus mutans, Cryptococcus neoformans, Klebsiella, Staphylococcus aureus, and Pseudomonas aeruginosa. In addition, as a carrier, the polypeptides may be used to induce immunity toward abnormal polysaccharides of tumor cells, thereby to produce anti-tumor antibodies for chemotherapy or diagnosis.

Also within the scope of this invention is a diagnosing method using the above-described antibodies or polypeptides containing SEQ ID NO: 3. Presence of the polypeptides or antibodies in a subject indicates that the subject is infected with a coronavirus. To detect the antibodies or polypeptides, one can obtain a test sample from a subject and detect the presence or absence of the antibodies or polypeptides using standard techniques, including ELISAs, immunoprecipitations, immunofluorescence, EIA, RIA, and Western blotting analysis.

The nucleic acid of this invention is useful as a hybridization probe for identifying coronavirus, e.g., SARS CoV, in a sample. The sample can be a clinical sample, including exudates, body fluids (e.g., serum, amniotic fluid, middle ear effusion, sputum, bronchoalveolar lavage fluid) and tissues. A variety of hybridization conditions may be employed to achieve varying degrees of selectivity of the probe toward the target sequences. A high degree of selectivity requires stringent conditions, such as conditions for hybridization in 6× sodium chloride/sodium citrate (SSC) at about 45° C., followed by one or more washes in 0.2×SSC, 0.1% SDS at 50-65° C.

A hybridization reaction can be performed both in a solution or on a solid phrase. In a solid phase, a test sequence from a sample is affixed to a selected matrix or surface. The fixed nucleic acid is then subjected to specific hybridization with selected probes comprising the nucleic acid of the present invention under desired conditions. The selected conditions will depend on the particular circumstances based on the particular criteria required depending on, for example, on the G+C contents, type of target nucleic acid, source of nucleic acid, size of hybridization probe etc. Following washing of the hybridization surface to remove non-specifically bound probe molecules, specific hybridization is detected or quantified, by means of the label. The selected probe should be at least 18 bp and may be in the range of 30 bp to 90 bp long.

In addition, A small interference RNA (SiRNA) corresponding to the nucleotide sequences of the present invention comprising the sequence of SEQ ID NO: 3 can be useful to block SARS CoV replication in vivo.

A polypeptide of this invention can also be used in a screening method of identifying a compound for treating an infection with a coronavirus, e.g., SARS CoV. One method, as described in the Summary section above, includes incubating a test compound and a first polypeptide containing SEQ ID NO: 3; and determining binding between the test compound and the first polypeptide. The presence of the binding indicates that the compound is a candidate compound.

Alternatively, a screening method includes (1) contacting a polypeptide of this invention with a suitable cell, to which the coronavirus binds to; and (2) determining a binding level between the polypeptide and the cell the presence or absence of a test compound. The binding level in the presence of the test compound, if lower than that in the absence of the test compound, indicates that the test compound can be used to treat an infection with the coronavirus. Examples of the cell include VERO E6 cells, NIH3T3 cells, HeLa cells, BHK-21 cells, and COS-7 cells. One can also use other cells that are capable of binding to a coronavirus.

The above-described polypeptides and antibodies can be used for treating an infection with a coronavirus, e.g., SARS. The invention therefore features a method of treating SARS, e.g., by administering to a subject in need thereof an effective amount of a polypeptide, an antibody, or a compound of the invention. Subjects to be treated can be identified as having, or being at risk for acquiring, a condition characterized by SARS. This method can be performed alone or in conjunction with other drugs or therapy.

Thus, also within the scope of this invention is a pharmaceutical composition that contains a pharmaceutically acceptable carrier and an effective amount of a polypeptide, an antibody, or a compound of the invention. The pharmaceutical composition can be used to treat coronavirus infection, such as SARS. The pharmaceutically acceptable carrier includes a solvent, a dispersion medium, a coating, an antibacterial and antifungal agent, and an isotonic and absorption delaying agent.

In one in vivo approach, a composition of this invention (e.g., a composition containing a polypeptide, an antibody, or a compound of the invention) is administered to a subject. Generally, the antibody or the compound is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100.0 mg/kg. Wide variations in the needed dosage are to be expected in view of the variety of compositions available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the composition in a suitable delivery vehicle (e.g., polymeric microparticles or implantable devices) may increase the efficiency of delivery, particularly for oral delivery.

A pharmaceutical composition of the invention can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the composition with a solid carrier and a lubricant. Examples of solid carriers include starch and sugar bentonite. The composition can also be administered in a form of a hard shell tablet or a capsule containing a binder, e.g., lactose or mannitol, conventional filler, and a tableting agent. The pharmaceutical composition can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent.

The efficacy of a composition of this invention can be evaluated both in vitro and in vivo. Briefly, the composition can be tested for its ability to inhibit the binding between a coronavirus and its target cell in vitro. For in vivo studies, the composition can be injected into an animal (e.g., a mouse model) and its therapeutic effects are then accessed. Based on the results, an appropriate dosage range and administration route can be determined.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

### EXAMPLE 1

In this example, the gene encoding the S protein of SARS CoV was cloned. More specifically, mRNA was extracted from Vero E6 cells infected with SARS-CoV isolated from SARS patients using the QIAamp Viral RNA Mini Kit (Qiagen) according to the manufacture's instruction. The extracted mRNA was resuspended in a TE buffer (10 mM Tris [symbol] Cl/1 mM EDTA, pH 8.0) and used as a template in RT-PCR to amplify the sequence encoding amino acids 1-1255 and 268-1255 of the S protein (Sfull and S268, respectively). Oligo-dT-18 (5′-TTTTTTTTTTTTTTTTTT-3′) and the following two pairs of primers were used:

| | |
|---|---|
| | Corresponding to |
| | nucleotides of |
| Sequences | GenBank NC_004718 |

Pair 1

| | | |
|---|---|---|
| CoV21477pET: | 5′-CACCATgTTTATTTTCTTATTATTTCTTA-3′ | 21477-21494 |
| CoV25241pET: | 5′-TgTgTAATgTAATTTgACACCCTTgAg-3′ | 25241-25215 |
| Pair 2 | | |
| CoV268pET: | 5′-CACC-ATggAAAATggTACAATCACA-3′ | 22278-22295 |
| CoV25241pET: | 5′-TgTgTAATgTAATTTgACACCCTTgAg-3′ | 25241-25215 |

The PCRs were carried out with an initial denaturating step at 94° C. for 5 minutes followed by 30 cycles of denaturating (94° C. for 1 minute), annealing (50° C. for 1 minute), and extension (68° C. for 4 minute), with a final prolonged extension step (68° C. for 10 minutes). The amplified coding sequences were inserted into the pET101/D-TOPO vectors to generate the plasmids, pET101/D-TOPO-S full and pET101/D-TOPO-S268. The plasmids were sequenced to confirm the coding sequences.

### EXAMPLE 2

Recombinant S protein and its fragments were expressed and purified according to a method modified from that described in Wang et al., 2003, Clin. Diagn. Lab. Immunol. 10, 451-458.

Briefly, Escherichia coli BL21DE3 bacteria were transformed with the afore-mentioned plasmids. The transformed cells were grown to an optical density of 600 nm at 0.7 to 0.8 and then induced with 1 mM/ml of IPTG (isopropyl β-D-thiogalactoside).

After an induction of 3 hours, the cells were harvested and lysed in a sonication buffer containing 50 mM sodium phosphate (pH 8.0), 10 μM PMSF, 0.1% Tween-20, 100 mM KCl, 500 mM NaCl, and 1 mg/ml of lysozyme for 30 minutes prior to sonication. Following centrifugation at 12,000 rpm in a Sigma 3K12 centrifuge with a Nr. 12158 rotor, the pellets were resuspended in buffer B (8 M urea, 0.1 M sodium phosphate(pH 8.0), and 10 mM Tris) and stirred at room temperature for 1 hour. After centrifugation, the supernatant was purified using metal chelate affinity chromatography and the Ni2+-nitrilotriacetic acid complexes (NTA) (Qiagen). Briefly, 20 ml of the supernatant was passed through a 2-ml column of Ni2+-NTA agarose that had been prewashed with buffers B and F (6 M guanidine-HCl, 0.2 M acetic acid), and pre-equilibrated in buffer B. The column was then washed with 10 volume of buffers B and C. Protein was eluted with buffer D (8 M urea, 0.1 M sodium phosphate, 10 mM Tris, pH 5.9) and buffer E (8 M urea, 0.1 M sodium phosphate, 10 mM Tris, pH 4.5) in 500-μl fractions. Protein-containing fractions were identified by sodium dodecylsulfate-polyacrylamide gel electrophoresis (SDS-PAGE) and Coomassie blue staining. To renature the protein, a stepwise dialysis was performed at 4° C. against buffer B containing decreasing concentrations of urea (4.0, 2.0, 1.0, 0.5, 0.25, 0.125, and 0.05 M) and against buffer D (20 mM Hepes, pH 8.0, 20% glycerol, 0.1 M KCl, 0.2 mM EDTA, 0.5 mM PMSF, 0.5 mM DTT, and 0.2% Nonidet P-40) alone. After dialysis and brief centrifugation in an Sigma 3K12 centrifuge 5402 (14,000 rpm) for 5 minutes at 4° C., the supernatant was quickly frozen in liquid nitrogen and stored at −80° C. The recombinant protein was purified to near homogeneity, as confirmed by Western blot with mouse anti-histidine monoclonal antibody.

### EXAMPLE 3

Antisera against recombinant SARS-CoV S protein were prepared. Rabbits, mice, and rats were obtained from the Animal Center of the Institute of Preventive Medicine of the National Defense Medical Center, Taiwan. All animals were confirmed healthy by a licensed veterinarian. To generate antisera against the S protein, each of the animals were subcutaneously immunized with recombinant proteins (40, 15, or 5 μg/animal/time for each rabbit, rat, and mouse) three times at an interval of one month. For the first immunization, the recombinant proteins were mixed with the complete Freund's adjuvant. For the second and third immunization, the proteins were mixed with the incomplete Freund's adjuvants. One month after the last immunization, the animals were bled and the sera collected. After 2 hour of agglutination at room temperature, the sera were centrifuged at 3000×g for 10 minutes at 4° C. in a Sigma 3K12 centrifuge with a Nr. 12154 rotor. Antisera were collected and mixed with 50% glycerol and stored at −20° C. The polyclonal antibodies raised in rabbit, rat, and mouse sera against purified recombinant S protein fragments were tested and confirmed to recognize both the recombinant and the SARS coronavirus S proteins by Western blot, ELISA, and indirect immunofluorescent assay (IFA). It was found that rabbit anti-rSfull antiserum recognized the S glycoprotein in SARS-CoV-infected Vero E6 cell lysate. Antibodies against purified recombinant S proteins were measured for their interaction with SARS-CoV S protein by ELISA and confirmed a titer of above 51,200 (for rat and rabbit anti-rSfull IgG), 25,600 (for rat anti-rS268 IgG), 19,200 (for BALB/c anti-rSfull IgG), or 68,260 (for BALB/c anti-rS268 IgG) after the last immunization. Rabbit anti-rSfull antisera and SARS patient serum also recognized SARS-CoV, while rabbit pre-immune serum did not. Finally, it was found that the rabbit anti-rSfull antisera captured the SARS-CoV S protein by heterogeneous antisera-based sandwich ELISA. Taken together, the antisera raised by recombinant S proteins were able to recognize and bind to the SARS-CoV S protein.

### EXAMPLE 4

Monoclonal antibodies were prepared. More specifically, BALB/c mice were injected received intraperitoneally (i.p.) 5 μg of recombinant S268 (rS268) in 100 ∥l of PBS emulsified with an equal volume of the complete Freund's adjuvant. After an interval of two weeks and four weeks, the mice were boosted with the protein in the same manner, except that the incomplete Freund's adjuvant was used. Three weeks after the third injection, final boosters containing 5 μg of antigen were injected i.p.

Hybridomas secreting anti-S antibodies were generated according to a standard procedure. Briefly, five days after the last injection, the spleen of an immunized mouse was removed and the splenocytes were fused with NSI/1-Ag4-1 (NS-1) myeloma cells. The splenocytes and the myeloma cells were washed twice with a serum-free RPMI solution, and mixed in a 15 ml conical tube. 1 ml 50% (v/v) polyethylene glycol (GIBCO BRL) was added to the mixture over a 1-minute period while the mixture was gently stirred. The mixture was then diluted by slowly (over 1 minute) adding 1 ml of DMEM twice followed by slowly adding (over 2 minutes) 8 ml of a serum-free DMEM medium. The mixture was centrifuged at 400×g for 5 minutes and the fused cell pellet was resuspended in an RPMI medium supplemented with 15% FBS, hypoxanthine-aminopterin-thymidine (HAT), and hybridoma cloning factor (ICN, Ohio, U.S.A.), and distributed (200 μl per well) in 96-well tissue culture plates. Hybridoma colonies were screened by enzyme-linked immunoadsorbent assay (ELISA) for secretion of MAbs that would bind to recombinant S protein. A number of cell clones were identified to secret anti-S protein antibodies (e.g., MAbs 1-13). Selected clones were subcloned by the limiting dilution method. Immunoglobulin classes and subclasses were determined using a subtyping kit (Roche Diagnostics, Penzberg, Germany). Ascitic fluids were produced in pristane-primed BALB/c mice to generate monoclonal antibodies in a large scale.

To further check the capability of the monoclonal antibodies to bind to recombinant S protein, an indirect immunofluorescent assay (IFA) (Kim et al., 2001, Microb. Pathog. 31, 145-15028) was performed. Briefly, Vero E6 monolayer cells infected with SARS-CoV were washed three times with PBS and then fixed in an acetone-Methanol mixture (1:1) for 3 minutes at room temperature. After blocking with 3% skim milk in PBS for 1 hour at room temperature, the monolayer cells were incubated for 1 hour at room temperature with monoclonal antibodies diluted in PBS containing 3% skim milk. After washing three times with PBS, the cells were incubated at room temperature with fluorescein isothiocyanate (FITC)-conjugated goat anti-human, goat anti-rabbit, and goat anti-mouse IgG diluted in PBS containing 3% skim milk for 1 hour, respectively. Finally, the monolayer cells were washed three times with PBS, and mounted with glycerol-PBS (1:1). The cells were viewed under an immunofluorescent microscope (LEICA, DMIRB). The results confirmed the capability of the afore-mentioned antibody to bind to the S protein expressed in the cells.

### EXAMPLE 5

ELISA was used to detect presence of anti-S protein antibodies in SARS patients. More specifically, 96-well microtiter plates (Falcon, #3912) were coated with purified recombinant S full-length protein (rSfull) or residues 268-1152 of the S protein (rS268) (50 μl/well, 0.2 mg/ml of 0.05 M carbonate buffer, pH 9.6) at 4° C. overnight. The contents of the plates were then discarded, and each well was filled with 200 μl PBS containing 3% skim-milk. The plates were then incubated for 1 hour at room temperature for blocking. After the contents were discarded, the wells were rinsed five times with PBST (PBS+0.05% Tween-20).

Diluted patient sera (S4, #0612, S45, S25, S18, Taichung-Shi, and Yanming-Yea), animal antisera, or control sera (S3, S31 and #284) were added into the wells respectively and incubated at 37° C. for 30 minutes. After being rinsed five times with PBST, the wells were incubated with 50 μl/well secondary antibodies at room temperature for 30 minutes. The secondary antibodies, including goat anti-rabbit IgG or IgM, goat anti-human IgG, and goat anti-mouse IgG, were conjugated with horseradish peroxidase and diluted 1:3000 in PBS containing 3% skim milk. The wells were then rinsed in the manner described above and reacted with a substrate solution (TMB, 50 mg/ml in phosphate-citrate buffer, pH 5.0) containing 1/1000 volume of 35% H₂O₂) at room temperature for 10 minutes. After the reaction was stopped by adding 1 M H₂SO₄ (50 μl/well), the optical densities (ODs) at 450 nm were measured. All sera were assayed in duplicate. Each plate included an air blank, as well as a negative control and a positive control. It was found that both rSfull and rS268 recombinant proteins were recognized by IgG of the SARS patients (S4, #0612, S45, S25, S18, Taichung-Shi, and Yanming-Yea), but not by the control sera (S3, S31 and #284). There results indicated that the S recombinant protein-based ELISA could be used to detect SARS-specific IgG in SARS-CoV infected patients.

Background reactivity and possible cross-reactivity were assessed by analyzing pre-immune serum specimens from healthy rabbits, mice, and rats. The cutoff values were set at ODn+3 SD, where ODn is the mean of ODs recorded or the pre-immune serum or mock specimens. This method was used for all investigations described below, with ODs greater than the calculated threshold ODs regarded as positive sera and all others regarded as negative.

### EXAMPLE 6

Sandwich ELISA were used to detect presence of the S protein in a sample. Briefly, 96-well microtiter plates (Falcon, #3912) were coated with rabbit pre-immune or anti-rSfull antisera (50 μl/well, 1/500 in 0.05 M carbonate buffer, pH 9.6) at 4° C. overnight. After the contents of the plates were discarded, the wells were blacked with PBS containing 3% skim milk (200 μl/well) 1 hour at room temperature. After being rinsed five times with PBST, the plates were incubated with a mock control (3% skim milk in PBST), rSfull (1:50˜1:800), rS268 (1:50˜1:800), Vero cell lysate (1:25˜1:800), and viral lysate (1:25˜1:800) at 37° C. for 1 hour. After washing three times with PBST, the above-described BALB/c anti-rS268 antibodies (1/2000 in PBST) were added to the wells, and the mixtures incubated at 37° C. for 30 minutes. After three washing with PBST, the plates were incubated with goat anti-mouse IgG-conjugated with horseradish peroxidase (1:2000 in PBST) at 37° C. for 30 minutes. After discarding the contents and washing with PBST, the plates were reacted with a substrate solution (TMB, 100 mg/ml in phosphate-citrate buffer, pH 5.0, containing 1/1000 volume of 35% H₂O₂) at room temperature for 15 minutes. After the reaction was stopped by adding 1 M H₂SO₄ (50 μl/well), the optical densities (ODs) at 450 nm were measured. Each dilution was assayed in duplicate. Each plate included a row of wells for air blanks.

### EXAMPLE 7

Virus neutralization assays were performed to test the therapeutic efficacy of the above described rabbit, rat, and mouse antisera.

To prepare viruse stock solution for neutralization assays, Vero E6 cells were infected with SARS-CoV and incubated in 5% of CO₂ at 37° C. for three days. The TCID50 of the infectious virus stock was calculated by the method of Reed and Muench (1938) (LaBarre et al., 2001, J. Virol. Methods 96, 107-126), and 1×10⁷ TCID50 of the virus stock solution was aliquoted and stored at −70° C.

For virus neutralization testing, 2×10⁵ Vero E6 cells/ml was inoculated onto a 12-well tissue culture plate (Falcon #3043, 96-well flexible plate) at 37° C. in 5% of CO₂ overnight. Pre-immune serum and antiserum of BALB/c mouse, rat, and rabbit raised against the recombinant S protein were pre-treated at 56° C. for 30 minutes to destroy heat-labile, nonspecific viral inhibitory substances. The sera were then diluted to the beginning dilution of 1/20 with a DMEM maintenance medium, and then added into a well containing 2×10⁴ TCID50 of the virus in a volume of 0.15 ml. Equal volumes of the serum and the test-virus dilutions were mixed and incubated at 37° C. for 1 hour. The serum-virus mixtures and virus controls (no sera) were inoculated into Vero E6-containing culture plates, which had been pre-washed with the DMEM maintenance medium and emptied just prior to the addition of serum-virus mixtures. After absorption at 37° C. for 2 hours, the wells were washed with the DMEM maintenance medium and then incubated with a 2% fetal calf serum/DMEM buffer for 24 hours at 37° C. The wells were washed twice with PBS (pH 7.4) before a lyses buffer was added. The microplates were stored at −70° C. or subjected to an SDS-PAGE/Western blot analysis as described below to examine the presence of virus replication. To confirm the neutralization effect, equal amounts of virus-infected cell lysates ( 1/10 of total lysates, 10μl) were also studied.

Each of the lysates was boiled in a sample buffer (125 mM Tris-HCl, pH 6.8, 100 mM DTT, 2% SDS, 20% glycerol, 0.005% brophenol blue) for 5 minutes, and then loaded onto an 8% SDS-polyacrylamide gel. After electrophoresis of SDS-PAGE, the proteins were transferred onto a Hybond-C extra membrane (Amersham Biosciences, Cat. No. RPN 303E) using a semidry apparatus (Amersham Biosciences, Large Semipllor Transphor Unit, Cat. No. 80-6211086). The membrane was blocked with a Blotto/Tween blocking buffer (5 mM Tris, pH 7.4; 77 mM NaCl; 0.05% Tween-20; 2.5% skim milk; and 0.001% antiform A) and then incubated with BALB/c anti-rS268 polyclonal antibody. The membrane was then washed with blocking buffer and further incubated with goat anti-mouse HRP-conjugated secondary antibody. The membrane was finally washed with blocking buffer and visualized with the ECL Western Blotting Detection Reagent (Amersham Biosciences, Cat. No. RPN2106). Viral protein levels were determined and compared to obtain the 90% virus neutralization titers.

Their virus neutralization titers of the above-mentioned antibodies were compared to those obtained from SARS-CoV patient's sera. It was found that the rabbit antisera were as effective as SARS-CoV patient sera in terms of virus neutralization in Vero E6 cells in a dose-dependent manner. By contrast, the rat and BALB/c mouse antisera had only modest neutralization effects to block SARS virus infection. The virus neutralization assay was also performed with monoclonal antibodies generated from BALB/c mice immunized with rS268. It was found that some of monoclonal antibodies (MAb 1, 3, 5, 7, and 8) had significant virus neutralizing effect; some (MAb 2, 4, and 6) had a little neutralizing effect; and some (MAb 9, 11, 12, and 13) had no neutralizing effect. These results revealed that the recombinant S protein conserved some antigenic epitope of SARS-CoV S glycoprotein and that antisera and monoclonal antibodies generated against the prokaryotic cell-expressed recombinant S protein fragment could neutralize SARS-CoV infection in Vero E6 cells.

### EXAMPLE 8

An entry-inhibition assay was performed to examine the entry of SARS-CoV into Vero E6 cells. Briefly, 2×10⁵ cells/ml of Vero E6 cells were inoculated into a 12-well tissue culture plate (Falcon #3043) and kept at 37° C. in 5% CO₂ overnight. SARS-CoV was diluted (1:4) with a DMEM maintenance medium to 2×10⁶ TCID50. Equal volumes (0.15 mL) of sera and test-virus dilutions were mixed and inoculated into Vero E6-containing culture plates, which had been pre-washed with the DMEM maintenance medium and emptied just prior to the addition of the serum-virus mixtures. After absorption at 4° C. for 2 hours, the wells were washed with the DMEM maintenance medium and incubated with 2% fetal calf serum DMEM at 37° C. for 24 hours. The wells were washed twice with PBS (pH 7.4), and a lyses buffer was added. The microplates were subjected to SDS-PAGE/Western blot analysis in the manner described above.

It was found that rabbit anti-rS full antisera inhibited the entry of SARS-CoV into Vero E6 cells, while a rabbit pre-immune antiserum and a SARS patient serum of SARS-CoV had only modest effects. Further, some of the above-described monoclonal antibodies (MAbs 1, 3, and 5) efficiently inhibited the entry of SARS-CoV into Vero E6 cells. Meanwhile, the capture of viral S protein by rabbit anti-rSfull antiserum depicted. By contrast, the rabbit pre-immune serum was not able to efficiently capture either recombinant or viral S proteins. These results indicated that rabbit antisera and monoclonal antibodies raised against prokaryotic cell-expressed recombinant S protein, could neutralize SARS-CoV infection in Vero E6 cells by inhibiting the entry of the virus into the cells.

### EXAMPLE 9

A systematic epitope-mapping assay was performed using synthetic peptides derived from S protein by ELISA to elucidate the mechanism of neutralization as well as to map a neutralizing epitope of the monoclonal antibodies. Some of the peptides were listed in the table below. Among them, CB116 to CB138, 15-mers with overlapping of 10 residues, covered residues 1128 to 1255 of the S protein.

| | | |
|---|---|---|
| Peptide | | |
| name | MW | Sequence |

S3 Fragment:

| | |
|---|---|
| S3-1 | GDSTECANLLLQYGS |
| S3-2 | LQYGSFCTQLNRALS |
| S3-3 | NRALSGIAAEQDRNT |
| S3-4 | QDRNTREVFAQVKQM |
| S3-5 | QVKQMYKTPTLKYFG |
| S3-6 | LKYFGGFNFSQILPD |
| S3-7 | QILPDPLKPTKRSFI |
| S3-8 | KRSFIEDLLFNKVTL |
| S3-9 | KVTLLADAGFMKQYG |
| S3-10 | MKQYGECLGDINARD |
| S3-11 | INARDLICAQKFNGL |
| S3-12 | KFNGLTVLPPLLTDD |
| S3-13 | LLTDDMIAAYTAALV |
| S3-14 | TAALVSGTATAGWTF |
| S3-15 | AGWTFGAGAALQIPF |
| S3-16 | LQIPFAMQMAYRFNG |
| S3-17 | YRFNGIGVTQNVLYE |
| S3-18 | NVLYENQKQIANQFN |
| S3-19 | ANQFNKAISQIQESL |
| S3-20 | IQESLTTTSTALGKL |
| S3-21 | ALGKLQDVVNQNAQA |
| S3-22 | QNAQALNTLVKQLSS |
| S3-23 | KQLSSNFGAISSVLN |
| S3-24 | SSVLNDILSRLDKVEA |
| S3-25 | LDKVEAEVQIDRLITG |
| S3-26 | RLITGRLQSLQTYVTQQLIRA |
| HR1 | ISSVLNDILSRLDKVEAEVQIDRLITGRLQSLQT |
| | YVTQQLIRAAEI |

S TM Fragment (CB peptide series)

| | | |
|---|---|---|
| 116 | 1,883.3 | DSFKEELDKYFKNHT |
| 117 | 1,790.1 | ELDKYFKNHTSPDVD |
| 118 | 1,627.0 | FKNHTSPDVDLGDIS |
| 119 | 1,441.8 | SPDVDLGDISGINAS |
| 120 | 1,481.8 | LGDISGINASVVNIQ |
| 121 | 1,637.9 | GINASVVNIQKEIDR |
| 122 | 1,721.9 | VVNIQKEIDRLNEVA |
| 123 | 1,767.1 | KEIDRLNEVAKNLNE |
| 124 | 1,667.1 | LNEVAKNLNESLIDL |
| 125 | 1,696.1 | KNLNESLIDLQELGK |
| 126 | 1,794.1 | SLIDLQELGKYEQYI |
| 127 | 2,013.2 | QELGKYEQYIKWPWY |
| 128 | 2,060.4 | YEQYIKWPWYVWLGF |
| 129 | 1,831.5 | KWPWYVWLGFIAGLI |
| 130 | 1,584.3 | VWLGFIAGLIAIVMV |
| 131 | 1,525.4 | IAGLIAIVMVTILLC |
| 132 | 1,583.4 | AIVMVTILLCCMTSC |
| 133 | 1,604.6 | TILLCCMTSCCSCLK |
| 134 | 1,482.4 | CMTSCCSCLKGACSC |
| 135 | 1,435.4 | CSCLKGACSCGSCCK |
| 136 | 1,522.1 | GACSCGSCCKFDEDD |
| 137 | 1,626.0 | GSCCKFDEDDSEPVL |
| 138 | 1673.0 | FDEDDSEPVLKGVKL |
| TM | | DSFKEELDRYFKNHTSPDVDLGDISGINASVV |
| HR2 | | DSFKEELDRYFKNHTSPDVDLGDISGINASVVIQ |
| | | KEIDRLNEVAKNLNESLIDLQELGKYEQYIK |
| SP1 | | NQCVNFNFNGLTGTGV |
| SP2 | | SSNFGAISSVLNDILSRLDKV |
| PEP508 | | GDYSHCSPLRYYPWWKCTYPDP |
| PEP509 | | TDAVDCSQNPLAELKCSVKSF |

Also generated were D1-TM; D2-TM, S1-Fc, S2, and S3. D1-TM was a bacterium-expressed fusion of the D1 region (aa 74 to 253 of the S protein) and TM region (aa 1130 to 1255 of the S protein), which were joined by a 8-Gly linker. D2-TM was a bacterium-expressed fusion of the D2 region (aa 294 to 739 of the S protein) and the TM region which were joined by a linker of 8 Gly. S1-Fc is a baculovirus expressed fusion of human Fc fragment of IgG1 and aa 1 to 333 of the S protein (S1). Bacterium-expressed S2, S3, and rRBD2 corresponded to aa 334-666, 667 to 999, and 294 to 739 of the S protein, respectively.

Microtiter plates were coated with different synthetic peptides (1 μg/well in a carbonate buffer) at 4° C. for overnight. These peptide included the just-mentioned D2-TM, S2-Fc, S3, rRBD2, D1 and D2 long peptide, CB116˜CB123, CB124˜131, CB132˜138, SP1, SP2, PEP508, PEP509, and NP (SARS CoV Urbani strain nucleocapside; sequence shown below)

MSDNGPQSNQ RSAPRTTFGG PTDSTDNNQN GGRNGARPKQ
RRPQGLPNNT ASWFTALTQH GKEELRFPRG QGVPINTNSG
PDDQIGYYRR ATRRVRGGDG KMKELSPRWY FYYLGTGPEA
SLPYGANKEG IVWVATEGAL NTPKDHIGTR NPNNNAATVL
QLPQGTTLPK GFYAEGSRGG SQASSRSSSR SRGNSRNSTP
GSSRGNSPAR MASGGGETAL ALLLLDRLNQ LESKVSGKGQ
QQQGQTVTKK SAAEASKKPR QKRTATKQYN VTQAFGRRGP
EQTQGNFGDQ DLIRQGTDYK HWPQIAQFAP SASAFFGMSR
IGMEVTPSGT WLTYHGAIKL DDKDPQFKDN VILLNKHIDA
YKTFPPTEPK KDKKKKTDEA QPLPQRQKKQ PTVTLLPAAD
MDDFSRQLQN SMSGASADST QA

The coated plates were then blocked for 1 hour and incubated with diluted neutralizing monoclonal antibodies 1-1, 3-2, 5-1, and 8-1 (1:5000 in PBS) at 37° C. for 1 hour. Bound monoclonal antibodies were incubated with goat anti-mouse IgG-HRP at 37° C. for 1 hour. After washing, ABT (Boehringer) or TMB substrate was added to each well and incubated for 30 minutes or 15 minutes at room temperature. The reactions were stopped with adding of 1N H₂SO₄, and specific SARS-CoV IgG was detected by OD405 nm reading. Each plate also included blanks and negative controls.

It was found that the neutralizing monoclonal antibodies (MAbs 1-1, 3-2, 5-1, and 8-1) bound to long synthetic peptides covering TM region (D1-TM and D2-TM). All of these neutralizing monoclonal antibodies bound to long peptide covering TM region (CB 116˜123). More detailed analysis revealed that all these neutralizing monoclonal antibodies specifically bound to CB119, which corresponded to a 15-amino acid sequence located at the tip of a heptad region (HR2) of the SARS-CoV S protein. All tested neutralizing monoclonal antibodies (MAbs 1, 2, 3-2, 5-1, 7-1, and 8-1) bound to CB 119 peptide. In contrast, the non-neutralizing monoclonal antibodies (4-2, 6-1, 9-1, 12-1, and 13-1) did not and failed to inhibit the entry of SARS-CoV into Vero E6 cells. These results indicated that binding of antibodies to the heptad region specifically and sufficiently neutralized the infection of VERO E6 cells by SARS-CoV, through blocking the entry of virus into cells.

An additional epitope mapping of the neutralizing monoclonal antibodies was conducted using FliTrx Random Peptide Display Library (Invitrogen, cat. no. K1125-01). A conserved peptide corresponding to amino acid residues within the region of residues 1143-1157 was defined. It suggests that the monoclonal antibodies neutralize the infection of SARS-CoV by blocking viral entry into cells, very likely through a fusion-inhibition mechanism.

### EXAMPLE 10

To utilize the above-described virus neutralization epitope, a synthtic peptide TM (DSFKEELDRYFKNHTSPDVDLGDISGINASVV) containing the sequence of the neutralization epitope was prepared and used to immunize rabbits. It was found that rabbit sera recognized the S protein, recombinant S protein fragments, and CB119 peptides. At 1/200 dilution, the rabbit sera could compete and inhibit the binding of MAbs 1, 3, and 5 to CB119 in competition ELISA. This result indicated that peptides containing the virus neutralization epitope are useful to generate antibodies to neutralize entry of SARS-CoV into host cells and thereby inhibit virus infections.

The above results suggest that infection of SARS-CoV could be inhibited by antisera against the virus neutralization epitope. Inhibition of SARS-CoV entry into Vero E6 cells was achieved using rabbit antiserum (1:50), but not the patient serum. This unexpected result indicated that the rabbit antiserum neutralized SARS-CoV infection through inhibition of the viral entry into Vero E6 cells, with a mechanism different for the SARS patient sera. An explanation for this difference is that the SARS patients produced neutralizing antibodies recognized other neutralization epitopes at different parts of S protein and no or little antibody recognized the tip of HR2 region as described above. Meanwhile, some anti-S monoclonal antibodies also had neutralization effect, but some of monoclonal antibodies had little or none.

The above results also revealed that prokaryotic cell-expressed S protein conserved epitopes related to viral entry into host cells; and antisera or monoclonal antibodies against this epitope sufficiently neutralized SARS-CoV infection through inhibition of the viral entry into Vero E6 cells. Recently, angiotensin-converting enzyme 2 (ACE2) is explored as a functional receptor for the SARS coronavirus in Vero E6 cells (Li et al., 2003, Nature 426, 450-454). ACE2 receptor-associated epitope on SARS-CoV S protein has also been identified (Sui et al., 2004, Proc. Natl. Acad. Sci. U.S.A. 101, 2536-2541). Neutralizing monoclonal antibodies described above can be used to find out alternative receptor-associated epitopes, as well as other conserved peptides which could inhibit the entry of virus into cells, by screening MAb-associated synthetic peptides mimics SARS-CoV S protein.

As described above, neutralizing monoclonal antibodies (MAbs 1-1, 3-2, 5-1, and 8-1) all interacted with a peptide containing residues mimicking heptad region (D1-TM, D2-TM, CB116˜119, or CB119), but not rRBD2, which contained a ACE2 receptor-interacting region. Further, all of the non-neutralizing monoclonal antibodies (MAbs 4-2, 6-1, 9-1, 12-1, and 13-1) did not react with peptide mimicking heptad region of S protein. This suggest that the monoclonal antibodies neutralize the infection of SARS-CoV through a pathway different from blocking the interaction between ACE2 receptor and SARS-CoV. Fusion of the membrane of enveloped viruses with the membrane of host cells is a prerequisite for viral entry into the cells. It is known that infections with coronaviruses, including MHV, FIPV, HCoV-229E, and HCoV-OC43, are achieved through fusion of the lipid bilayer of the viral envelop with host cell membranes (Bos et al., 1995, Virology 214, 453-463; De Groot et al., 1989 Virology 171 493-502; Luo et al., 1998, Virology 244, 483-294; Spaan et al., 1988, J. Gen. Virol. 69(Pt 12), 2939-2952; El-Sahly et al., 2000, Infect. Dis. 31, 96-100; and Folz et al., 1999, Chest 115, 901-905). Studies indicate that coronavirus S protein can be classified as a type 1 viral fusion protein (Bosch et al., 2003, J. Virol. 77, 8801-8811; and Tripet et al., 2004, J. Biol. Chem. 279, 20836-2084935-36). They show similar fusion process in the fusogenic state for the structures of the ectodomains, suggesting a general membrane fusion mechanism (Baker et al., 1999, Mol. Cell 3, 309-319; Caffrey et al., 1998, EMBO J. 17, 4572-4584; Chan et al., 1997, Cell 89, 263-273; Fass et al., 1996, Nat. Struct. Biol. 3, 465-469; Kobe et al., 1999, Proc. Natl. Acad. Sci. U.S.A. 96, 4319-4324; Malashkevich et al., 1998, Proc. Natl. Acad. Sci. U.S.A. 95, 9134-9139; Malashkevich et al., 1999, Proc. Natl. Acad. Sci. U.S.A. 96, 2662-2667; Tan et al., 1997, Proc. Natl. Acad. Sci. U.S.A. 94, 12303-1238; Weissenhorn, et al., 1998, Mol. Cell 2, 605-616; Weissenhorn et al., 1997, Nature 387, 426-430; and Yang et al., 1999. J. Struct. Biol. 126, 131-144). Further, the HR-N and HR-C regions of the SARS-CoV S glycoprotein independently form α-helical coiled-coil structures, and a mixture of HR-N and HR-C form a very stable trimer of dimmers structure similar to other type 1 viral fusion protein (Tripet et al., 2004, J. Biol. Chem. 279, 20836-2084936). In this study, neutralizing monoclonal antibodies all react with CB 119 peptide mimicking amino acid residues located at 1143-1157 of S protein, which overlapped with the interaction region (residues 1151-1185) of HR-C domain (Tripet et al., 2004, J. Biol. Chem. 279, 20836-2084936).

### EXAMPLE 11

An residue substitution assay was carried out to determine amino acids within the neutralization epitope (SEQ ID NO: 3; CB119) that were important for the monoclonal antibodies to bind to. Fifteen alanine substitution analogs of CB119 (i.e., CB119-2 to CB 119-16) were synthesized and shown in the table below. The substitution alanine or glycine residue in each analog was bold.

| | |
|---|---|
| CB119 analogs | Sequences |

| | |
|---|---|
| CB119-1 | SPDVDLGDISGINAS |
| CB119-2 | APDVDLGDISGINAS |
| CB119-3 | SADVDLGDISGINAS |
| CB119-4 | SPAVDLGDISGINAS |
| CB119-5 | SPDADLGDISGINAS |
| CB119-6 | SPDVALGDISGINAS |
| CB119-7 | SPDVDAGDISGINAS |
| CB119-8 | SPDVDLADISGINAS |
| CB119-9 | SPDVDLGAISGINAS |
| CB119-10 | SPDVDLGDASGINAS |
| CB119-11 | SPDVDLGDIAGINAS |
| CB119-12 | SPDVDLGDISAINAS |
| CB119-13 | SPDVDLGDISGANAS |
| CB119-14 | SPDVDLGDISGIAAS |
| CB119-15 | SPDVDLGDISGINGS |
| CB119-16 | SPDVDLGDTSGINAA |

The above polypeptides were coated onto an ELISA plate and peptide-ELISA was performed in the same manner described above. It was found that MAbs 1-1, 3-2, 5-1 and 8-1 bound significantly less to peptides CB119-6 and CB119-11. These results indicated that asparatic acid (D) residue and serine residue at amino acid positions 5 and 10, respectively, were essential for the binding by the monoclonal antibodies. These results also explained why the monoclonal antibodies did not bind to CB 118 (missing asparatic acid) and CB 120 (missing serine).

What is claimed is:

1. An isolated polypeptide comprising SEQ ID NO: 3, wherein the polypeptide is 15-100 amino acid residues in length.

2. The polypeptide of claim 1, wherein the polypeptide is 15-50 amino acid residues in length.

3. The polypeptide of claim 2, wherein the polypeptide is 15-32 amino acid residues in length.

4. The polypeptide of claim 1, wherein the polypeptide contains SEQ ID NO: 4.

5. An isolated nucleic acid comprising a sequence encoding a polypeptide of claim 1 or a complement thereof.

6. The nucleic acid of claim 5, wherein the nucleic acid contains SEQ ID NO: 5 or 6.

7. An expression vector comprising a nucleic acid of claim 5.

8. A host cell comprising a nucleic acid of claim 5.

9. A method of producing a polypeptide, comprising culturing the host cell of claim 8 in a medium under conditions permitting expression of a polypeptide encoded by the nucleic acid, and purifying the polypeptide from the cultured cell or the medium of the cell.

10. A composition comprising a polypeptide of claim 1 or a nucleic acid encoding the polypeptide; and a pharmaceutically acceptable carrier.

11. A method of generating an antibody against a polypeptide, the method comprising administering to a non-human animal a polypeptide of claim 1 or a nucleic acid encoding the polypeptide.

12. A purified antibody that binds specifically to a polypeptide containing SEQ ID NO: 3.

13. The antibody of claim 12, wherein the antibody is a monoclonal antibody.

14. The antibody of claim 13, wherein the monoclonal antibody is selected from the group consisting of MAb 1, MAb 2, MAb 3, MAb 4, MAb 5, MAb 6, MAb 7, MAb 8, MAb 9, MAb 10, MAb 11, MAb 12, MAb13, MAb 1-1, MAb 3-2, MAb 5-1, and MAb 8-1.

15. A method of inducing an immune response in a subject against a coronavirus, the method comprising administering to the subject a polypeptide of claim 1 or a nucleic acid encoding the polypeptide.

16. The method of claim 15, wherein the coronavirus is a SARS coronavirus.

17. A method of diagnosing an infection with a coronavirus in a subject, comprising:
i) providing a test sample from a subject, and
ii) determining presence of a polypeptide containing SEQ ID NO: 3 or an antibody that binds thereto,
wherein presence of the polypeptide or the antibody in the test sample indicates the subject is infected with the coronavirus.

18. The method of claim 17, wherein the test sample is a blood sample.

19. The method of claim 17, wherein the polypeptide contains SEQ ID NO:4.

20. The method of claim 17, wherein the coronavirus is a SARS coronavirus.

21. A method of treating an infection with a coronavirus, the method comprising administering to a subject in need thereof an effective amount of an antibody of claim 12.

22. The method of claim 21, wherein the coronavirus is a SARS coronavirus.

23. A kit for detecting presence of a coronavirus in a sample, comprising a polypeptide containing SEQ ID NO: 3 or an antibody that binds specifically thereto.

24. The kit of claim 23, wherein the coronavirus is a SARS coronavirus.

25. A screening method of identifying a candidate compound for treating an infection with a coronavirus, the method comprising:
incubating a test compound and a first polypeptide containing SEQ ID NO: 3; and
determining binding between the test compound and the first polypeptide, wherein the presence of the binding indicates that the compound is a candidate compound.

26. The method of claim 25, wherein the coronavirus is a SARS coronavirus.

27. The screening method of claim 25, further comprising
incubating the test compound and a second polypeptide, the second polypeptide being identical to the first polypeptide except that Asp₅ of SEQ ID NO: 3 is replaced with a non-Asp residue or Ser₁₀ of SEQ ID NO: 3 is replaced with a non-Ser residue; and
determining binding between the test compound and the second polypeptide, wherein the compound is determined to be a candidate compound if the binding between the test compound and the first polypeptide is stronger than the binding between the test compound and the second polypeptide.

28. The screening method of claim 25, wherein the compound is an antibody.

29. The screening method of claim 25, wherein the coronavirus is a SARS coronavirus.


SEQUENCE LISTING (82 sequences)

SEQ ID NO: 1 - protein, 1255 aa, SARS coronavirus Urbani
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - DNA, 3768 bp, SARS coronavirus Urbani
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 3 - protein, 15 aa, Artificial Sequence
  Synthetically generated peptide
SPDVDLGDISGINAS

SEQ ID NO: 4 - protein, 32 aa, Artificial Sequence
  Synthetically generated peptide
DSFKEELDRYFKNHTSPDVDLGDISGINASVV

SEQ ID NO: 5 - DNA, 45 bp, Artificial Sequence
  Synthetically generated oligonucleotide
tcaccagatgttgatcttggcgacatttcaggcattaacgcttct

SEQ ID NO: 6 - DNA, 96 bp, Artificial Sequence
  Synthetically generated oligonucleotide
gactcattcaaagaagagctggacaagtacttcaaaaatcatacatcaccagatgttgatcttggcgacatttcaggcattaacgcttctgtcgtc

SEQ ID NO: 7 - DNA, 18 bp, Artificial Sequence
  Synthetically generated oligonucleotide
tttttttttttttttttt

SEQ ID NO: 8 - DNA, 29 bp, Artificial Sequence
  Primer
caccatgtttattttcttattatttctta

SEQ ID NO: 9 - DNA, 27 bp, Artificial Sequence
  Primer
tgtgtaatgtaatttgacacccttgag

SEQ ID NO: 10 - DNA, 25 bp, Artificial Sequence
  Primer
caccatggaaaatggtacaatcaca

SEQ ID NO: 11 - protein, 15 aa, Artificial Sequence
  Synthetically generated peptide
GDSTECANLLLQYGS

SEQ ID NO: 12 - protein, 15 aa, Artificial Sequence
  Synthetically generated peptide
LQYGSFCTQLNRALS

SEQ ID NO: 13 - protein, 15 aa, Artificial Sequence
  Synthetically generated peptide
NRALSGIAAEQDRNT

SEQ ID NO: 14 - protein, 15 aa, Artificial Sequence
  Synthetically generated peptide
QDRNTREVFAQVKQM

SEQ ID NO: 15 - protein, 15 aa, Artificial Sequence
  Synthetically generated peptide
QVKQMYKTPTLKYFG

SEQ ID NO: 16 - protein, 15 aa, Artificial Sequence
  Synthetically generated peptide
LKYFGGFNFSQILPD

SEQ ID NO: 17 - protein, 15 aa, Artificial Sequence
  Synthetically generated peptide
QILPDPLKPTKRSFI

SEQ ID NO: 18 - protein, 15 aa, Artificial Sequence
  Synthetically generated peptide
KRSFIEDLLFNKVTL

SEQ ID NO: 19 - protein, 15 aa, Artificial Sequence
  Synthetically generated peptide
KVTLLADAGFMKQYG

SEQ ID NO: 20 - protein, 15 aa, Artificial Sequence
  Synthetically generated peptide
MKQYGECLGDINARD

SEQ ID NO: 21 - protein, 15 aa, Artificial Sequence
  Synthetically generated peptide
INARDLICAQKFNGL

SEQ ID NO: 22 - protein, 15 aa, Artificial Sequence
  Synthetically generated peptide
KFNGLTVLPPLLTDD

SEQ ID NO: 23 - protein, 15 aa, Artificial Sequence
  Synthetically generated peptide
LLTDDMIAAYTAALV

SEQ ID NO: 24 - protein, 15 aa, Artificial Sequence
  Synthetically generated peptide
TAALVSGTATAGWTF

SEQ ID NO: 25 - protein, 15 aa, Artificial Sequence
  Synthetically generated peptide
AGWTFGAGAALQIPF

SEQ ID NO: 26 - protein, 15 aa, Artificial Sequence
  Synthetically generated peptide
LQIPFAMQMAYRFNG

SEQ ID NO: 27 - protein, 15 aa, Artificial Sequence
  Synthetically generated peptide
YRFNGIGVTQNVLYE

SEQ ID NO: 28 - protein, 15 aa, Artificial Sequence
  Synthetically generated peptide
NVLYENQKQIANQFN

SEQ ID NO: 29 - protein, 15 aa, Artificial Sequence
  Synthetically generated peptide
ANQFNKAISQIQESL

SEQ ID NO: 30 - protein, 15 aa, Artificial Sequence
  Synthetically generated peptide
IQESLTTTSTALGKL

SEQ ID NO: 31 - protein, 15 aa, Artificial Sequence
  Synthetically generated peptide
ALGKLQDVVNQNAQA

SEQ ID NO: 32 - protein, 15 aa, Artificial Sequence
  Synthetically generated peptide
QNAQALNTLVKQLSS

SEQ ID NO: 33 - protein, 15 aa, Artificial Sequence
  Synthetically generated peptide
KQLSSNFGAISSVLN

SEQ ID NO: 34 - protein, 16 aa, Artificial Sequence
  Synthetically generated peptide
SSVLNDILSRLDKVEA

SEQ ID NO: 35 - protein, 16 aa, Artificial Sequence
  Synthetically generated peptide
LDKVEAEVQIDRLITG

SEQ ID NO: 36 - protein, 21 aa, Artificial Sequence
  Synthetically generated peptide
RLITGRLQSLQTYVTQQLIRA

SEQ ID NO: 37 - protein, 46 aa, Artificial Sequence
  Synthetically generated peptide
ISSVLNDILSRLDKVEAEVQIDRLITGRLQSLQTYVTQQLIRAAEI

SEQ ID NO: 38 - protein, 15 aa, Artificial Sequence
  Synthetically generated peptide
DSFKEELDKYFKNHT

SEQ ID NO: 39 - protein, 15 aa, Artificial Sequence
  Synthetically generated peptide
ELDKYFKNHTSPDVD

SEQ ID NO: 40 - protein, 15 aa, Artificial Sequence
  Synthetically generated peptide
FKNHTSPDVDLGDIS

SEQ ID NO: 41 - protein, 15 aa, Artificial Sequence
  Synthetically generated peptide
SPDVDLGDISGINAS

SEQ ID NO: 42 - protein, 15 aa, Artificial Sequence
  Synthetically generated peptide
LGDISGINASVVNIQ

SEQ ID NO: 43 - protein, 15 aa, Artificial Sequence
  Synthetically generated peptide
GINASVVNIQKEIDR

SEQ ID NO: 44 - protein, 15 aa, Artificial Sequence
  Synthetically generated peptide
VVNIQKEIDRLNEVA

SEQ ID NO: 45 - protein, 15 aa, Artificial Sequence
  Synthetically generated peptide
KEIDRLNEVAKNLNE

SEQ ID NO: 46 - protein, 15 aa, Artificial Sequence
  Synthetically generated peptide
LNEVAKNLNESLIDL

SEQ ID NO: 47 - protein, 15 aa, Artificial Sequence
  Synthetically generated peptide
KNLNESLIDLQELGK

SEQ ID NO: 48 - protein, 15 aa, Artificial Sequence
  Synthetically generated peptide
SLIDLQELGKYEQYI

SEQ ID NO: 49 - protein, 15 aa, Artificial Sequence
  Synthetically generated peptide
QELGKYEQYIKWPWY

SEQ ID NO: 50 - protein, 15 aa, Artificial Sequence
  Synthetically generated peptide
YEQYIKWPWYVWLGF

SEQ ID NO: 51 - protein, 15 aa, Artificial Sequence
  Synthetically generated peptide
KWPWYVWLGFIAGLI

SEQ ID NO: 52 - protein, 15 aa, Artificial Sequence
  Synthetically generated peptide
VWLGFIAGLIAIVMV

SEQ ID NO: 53 - protein, 15 aa, Artificial Sequence
  Synthetically generated peptide
IAGLIAIVMVTILLC

SEQ ID NO: 54 - protein, 15 aa, Artificial Sequence
  Synthetically generated peptide
AIVMVTILLCCMTSC

SEQ ID NO: 55 - protein, 15 aa, Artificial Sequence
  Synthetically generated peptide
TILLCCMTSCCSCLK

SEQ ID NO: 56 - protein, 15 aa, Artificial Sequence
  Synthetically generated peptide
CMTSCCSCLKGACSC

SEQ ID NO: 57 - protein, 15 aa, Artificial Sequence
  Synthetically generated peptide
CSCLKGACSCGSCCK

SEQ ID NO: 58 - protein, 15 aa, Artificial Sequence
  Synthetically generated peptide
GACSCGSCCKFDEDD

SEQ ID NO: 59 - protein, 15 aa, Artificial Sequence
  Synthetically generated peptide
GSCCKFDEDDSEPVL

SEQ ID NO: 60 - protein, 15 aa, Artificial Sequence
  Synthetically generated peptide
FDEDDSEPVLKGVKL

SEQ ID NO: 61 - protein, 42 aa, Artificial Sequence
  Synthetically generated peptide
DSFKEELDRYFKNHTSPDVDLGDISGINASVVIQKEIDRLNE

SEQ ID NO: 62 - protein, 23 aa, Artificial Sequence
  Synthetically generated peptide
VAKNLNESLIDLQELGKYEQYIK

SEQ ID NO: 63 - protein, 16 aa, Artificial Sequence
  Synthetically generated peptide
NQCVNFNFNGLTGTGV

SEQ ID NO: 64 - protein, 21 aa, Artificial Sequence
  Synthetically generated peptide
SSNFGAISSVLNDILSRLDKV

SEQ ID NO: 65 - protein, 22 aa, Artificial Sequence
  Synthetically generated peptide
GDYSHCSPLRYYPWWKCTYPDP

SEQ ID NO: 66 - protein, 21 aa, Artificial Sequence
  Synthetically generated peptide
TDAVDCSQNPLAELKCSVKSF

SEQ ID NO: 67 - protein, 422 aa, Artificial Sequence
  Synthetically generated peptide
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 68 - protein, 15 aa, Artificial Sequence
  Synthetically generated peptide
APDVDLGDISGINAS

SEQ ID NO: 69 - protein, 15 aa, Artificial Sequence
  Synthetically generated peptide
SADVDLGDISGINAS

SEQ ID NO: 70 - protein, 15 aa, Artificial Sequence
  Synthetically generated peptide
SPAVDLGDISGINAS

SEQ ID NO: 71 - protein, 15 aa, Artificial Sequence
  Synthetically generated peptide
SPDADLGDISGINAS

SEQ ID NO: 72 - protein, 15 aa, Artificial Sequence
  Synthetically generated peptide
SPDVALGDISGINAS

SEQ ID NO: 73 - protein, 15 aa, Artificial Sequence
  Synthetically generated peptide
SPDVDAGDISGINAS

SEQ ID NO: 74 - protein, 15 aa, Artificial Sequence
  Synthetically generated peptide
SPDVDLADISGINAS

SEQ ID NO: 75 - protein, 15 aa, Artificial Sequence
  Synthetically generated peptide
SPDVDLGAISGINAS

SEQ ID NO: 76 - protein, 15 aa, Artificial Sequence
  Synthetically generated peptide
SPDVDLGDASGINAS

SEQ ID NO: 77 - protein, 15 aa, Artificial Sequence
  Synthetically generated peptide
SPDVDLGDIAGINAS

SEQ ID NO: 78 - protein, 15 aa, Artificial Sequence
  Synthetically generated peptide
SPDVDLGDISAINAS

SEQ ID NO: 79 - protein, 15 aa, Artificial Sequence
  Synthetically generated peptide
SPDVDLGDISGANAS

SEQ ID NO: 80 - protein, 15 aa, Artificial Sequence
  Synthetically generated peptide
SPDVDLGDISGIAAS

SEQ ID NO: 81 - protein, 15 aa, Artificial Sequence
  Synthetically generated peptide
SPDVDLGDISGINGS

SEQ ID NO: 82 - protein, 15 aa, Artificial Sequence
  Synthetically generated peptide
SPDVDLGDISGINAA